(19) United States
(12) Patent Application Publication
(10) Pub. No.: US 2011/0137165 A1
(43) Pub. Date: Jun. 9, 2011

(54) ULTRASOUND IMAGING

(75) Inventors: Cecile Dufour, Paris (FR); Olivier Gerard, Viroflay (FR); Thomas Gauthier, Seattle, WA (US)

(73) Assignee: KONINKLIJKE PHILIPS ELECTRONICS N.V., Eindhoven (NL)

(21) Appl. No.: 13/056,144

(22) PCT Filed: Aug. 7, 2009
(86) PCT No.: PCT/IB09/53486

§ 371 Date: Jan. 27, 2011

(51) Int. Cl. A61B 8/00

(52) U.S. Cl. 600/437

(57) ABSTRACT

In an ultrasound imaging system (UIS), an image capturing arrangement (ICA) captures a sequence of ultrasound images (IMS) of a body (BDY) while an object (NDL) is introduced into the body. A displacement detector (DD) generates a map of displacement indications (DM) from the sequence of ultrasound images. A displacement indication relates to a particular portion of the body and indicates a displacement that the portion has undergone. An object locator (OL) provides an indication (OLI) relating to the location of the object in the body on the basis of the map of displacement indications.

### FIELD OF THE INVENTION

An aspect of the invention relates to a method of ultrasound imaging. The method may be used, for example, to provide visual information pertaining to an object that is introduced into a body. The visual information may indicate a current location of the object within the body, or a current direction in which the object moves within the body, or both. Other aspects of the invention relate to an ultrasound imaging arrangement, and a computer program product.

### BACKGROUND OF THE INVENTION

Ultrasound imaging typically involves the following operations. A probe that comprises piezoelectric transducers is held against a body that needs to be examined. A transmitter circuit generates respective activation signals that are applied to respective piezoelectric transducers of the probe. This causes the probe to emit ultrasound waves into a body, typically in the form of acoustic beams. Reflections of the ultrasound waves occur within the body. At least a portion of these reflected waves travel back to the probe. This causes respective piezoelectric transducers to produce respective reception signals. A receiver circuit processes these reception signals so as to obtain an ultrasound image of the body.

It is desirable that ultrasound images provide useful visual feedback in case an operator introduces an object into a body. The ultrasound images may guide the operator in moving the object to a particular region of interest in the body. For example, ultrasound images may potentially guide a clinician who introduces a needle into the body of a patient. Accordingly, it can be avoided that several trials and errors are needed before the clinician succeeds in reaching the particular region of interest. Such trials and errors cause patient discomfort and, moreover, are time consuming for the clinician.

However, it is generally difficult to accurately track an object that has been introduced into a body by means of ultrasound imaging. Ultrasound images typically provide structural details of body portions that lie in a given plane or in a given set of planes, which are typically referred to as view planes. A view plane may be regarded as a particular cross-section of the body of which a photo, or rather a film, is made. In case of two-dimensional (2-D) ultrasound imaging, there is one view plane that has a particular orientation corresponding with that of the acoustic beams. In case of three-dimensional (3-D) ultrasound imaging, there are several view planes of different orientation.

Whatever the ultrasound imaging technique that is used, 2-D or 3-D, it holds that body portions that lie outside a view plane are not represented by that view plane. Consequently, in case there is not any view plane that precisely matches the object that is introduced into the body, or at least a substantial portion thereof, the object will be hardly visible or not visible at all. A view plane may be adjusted in a manual fashion by manipulating the probe or in an electrical fashion by appropriate processing in the transmitter circuit or the receiver circuit, or both. However, in order to correctly adjust a view plane, some positional information about the object is required. Obtaining this information may be relatively time consuming if, for example, a search procedure is applied, or may involve relatively costly devices, or both.

United States patent application published under number U.S. 2007/0167769 describes an ultrasonic diagnosis apparatus that allows displaying a path of insertion of a puncture needle. Ultrasonic volume data is created based by means of an ultrasonic probe, which three-dimensionally scans a living body. A tomographic plane is selected from the ultrasonic volume data for display on a display device. In a first embodiment, this plane selection is done manually. An operator first has to designate two points in the ultrasonic volume data: one point corresponding with a basal part of the puncture needle, the other point corresponding with a tip part of the puncture needle. The operator has to manually select respective two-dimensional images from the ultrasonic volume data in order to visualize the aforementioned parts of the puncture needle, which need to be designated. Subsequently, the operator selects the tomographic plane of interest by designating an angle of rotation around an axis, which is a straight line through the aforementioned two points. In a second embodiment, the plane selection is based on position information provided by a position detection arrangement, which detects the position of the ultrasonic probe and a therapeutic device that includes the puncture needle.

### SUMMARY OF THE INVENTION

There is a need for an improved ultrasound imaging technique, which provides information pertaining to an object that is introduced into a body.

In accordance with an aspect of the invention, a sequence of ultrasound images of a body is captured while an object is introduced into the body. A map of displacement indications is generated from the sequence of ultrasound images. A displacement indication relates to a particular portion of the body and indicates a displacement that the portion has undergone. An indication relating to the location of the object in the body is provided on the basis of the map of displacement indications.

A current location of the object, as well as a current direction that the object follows, determine to a relatively large extent respective displacements that respective portions of the body undergo. The map of displacement indications reflects these respective displacements. Consequently, information about the current location of the object, as well as its current direction, can be extracted from this map. For example, a body portion that undergoes a relatively large displacement is typically located relatively close to the object that has been introduced into the body. A line along which respective displacements have similar orientations is likely to correspond with the current direction of the object. A section along this line that exhibits a steep decrease in displacement magnitude will typically correspond with a tip portion of the object of interest.

There is no need for a three-dimensional scan of the body in order to obtain information about the current location of the object or its current direction. A two-dimensional scan is sufficient, although a three-dimensional scan can be used. Moreover, there is no need for an operator to search and designate portions of the object in different view planes so as to determine a view plane that matches the object. Neither is there any need for particular devices that detect the location of the object in the body. Accordingly, the present invention provides a low-cost ultrasound imaging technique, which provides information pertaining to an object that is introduced into a body. Moreover, this ultrasound imaging technique is user-friendly and time efficient.

An implementation of the invention advantageously comprises one or more of the following additional features, which are described in separate paragraphs that correspond with individual dependent claims.

Preferably, a display image is formed that comprises an ultrasound image and a visual indication, which is based on the indication relating to the location of the object in the body obtained as defined hereinbefore.

Preferably, an axis of symmetry is identified in the map of displacement indications.

Preferably, a display image is formed that comprises an ultrasound image and a visual indication of a direction in which the object moves within the body, the visual indication being based on the axis of symmetry.

Preferably, a steep decrease in magnitude of displacement indications along the axis of symmetry is identified.

Preferably, a display image is formed that comprises an ultrasound image and a visual indication of a tip portion of the object, the visual indication being based on the steep decrease in magnitude of displacement indications along the axis of symmetry.

In case a three-dimensional scan of the body that produces volume data is carried out, a view plane that coincides with the object introduced into the body is generated from the volume data on the basis of the indication relating to the location of the object in the body. A display image may be formed that comprises this view plane.

Preferably, the map of displacement indications is obtained as follows. A map of elementary displacement indications is generated from a pair of ultrasound images, which are temporarily neighboring. An elementary displacement indication links a particular location in one image of a pair to a particular location in the other image. A map of accumulated displacement indications is generated on the basis of respective maps of elementary displacement indications generated from respective pairs of ultrasound images. An accumulated displacement indication corresponds to a sum of respective elementary displacement indications that link respective image locations in respective images.

The map of elementary displacement indications and the map of accumulated displacement indications may be generated on an image by image basis. In that case, a recent version of the map of accumulated displacement indications, which has previously been generated, is read from a memory. Respective elementary displacement indications that are generated from a pair of images are applied to corresponding respective accumulated displacement indications comprised in the map of accumulated displacement indications, which has been read from the memory. Accordingly, an updated version of the map of accumulated displacement indications is obtained. The updated version is then written into a memory.

The accumulated displacement indications may be expressed as respective points associated with respective locations in an initial image. These respective points are shifted in terms of image location as a result of respective elementary displacement indications that have been established.

A detailed description, with reference to drawings, illustrates the invention summarized hereinbefore as well as the additional features.

### BRIEF DESCRIPTION OF THE DRAWINGS

FIG. 1 is a block diagram that illustrates an ultrasound imaging system.

FIG. 2 is a block diagram that illustrates a displacement detector, which forms part of the ultrasound imaging system.

FIGS. 3-11 are conceptual diagrams that illustrate a mode of operation of the displacement detector.

FIG. 12 is a data diagram that illustrates a vector-based version of the displacement map, which the displacement detector may provide.

FIG. 13 is a data diagram that illustrates a grid point-based version of the displacement map, which the displacement detector may provide.

FIG. 14 is a pictorial diagram that illustrates a 2-D mode display image, which the ultrasound imaging system may provide based on a two-dimensional ultrasound scan.

FIG. 15 is a pictorial diagram that illustrates a 3-D mode display image, which the ultrasound imaging system may provide based on a three-dimensional ultrasound scan.

### DETAILED DESCRIPTION OF THE INVENTION

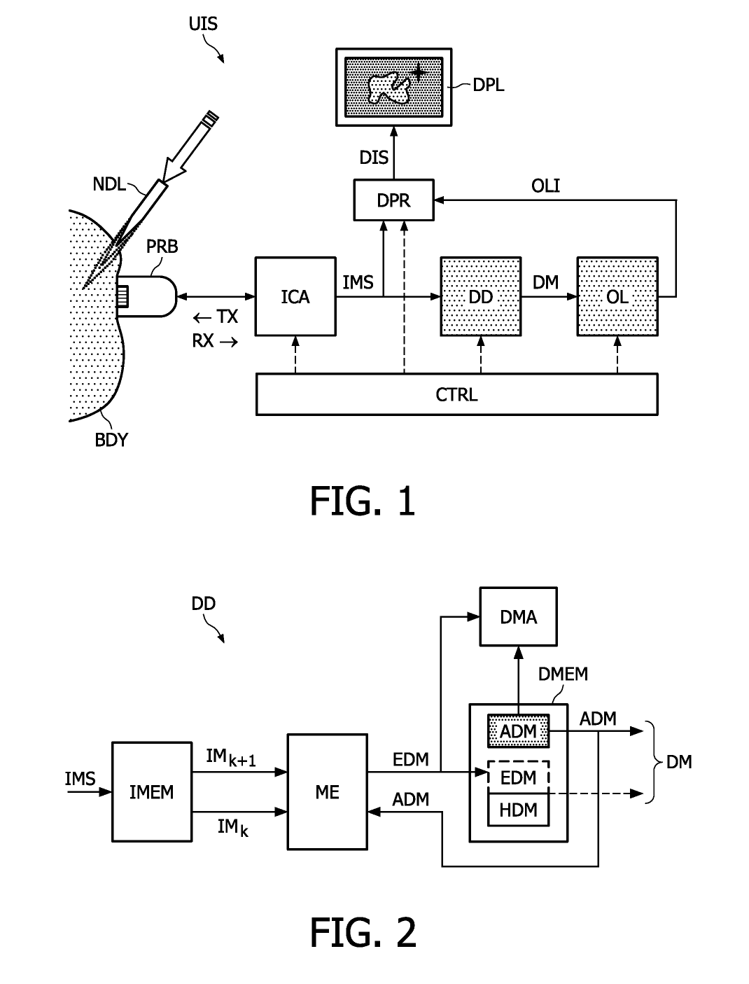

FIG. 1 illustrates an ultrasound imaging system UIS, which may assist a clinician in appropriately inserting a needle NDL into a body BDY of a patient. The ultrasound imaging system UIS comprises a probe PRB, an image capturing arrangement ICA, a display processor DPR, a display device DPL, and a controller CTRL. The probe PRB may comprise, for example, a two-dimensional array of piezoelectric transducers. The image capturing arrangement ICA may comprise an ultrasound transmitter and an ultrasound receiver, which may include a beam-forming module. The image capturing arrangement ICA may further comprise one or more filter modules and a so-called B-mode processing module. The controller CTRL may be in the form of, for example, a suitably programmed processor. The controller CTRL may further comprise a user interface, which is not illustrated for reasons of convenience.

The ultrasound imaging system UIS further comprises the following functional entities: a displacement detector DD and an object locator OL. These functional entities may each be implemented by means of, for example, a set of instructions that have been loaded into a programmable processor. In such a software-based implementation, the set of instructions defines operations that the functional entity concerned carries out, which will be described hereinafter. FIG. 1 can thus be regarded to represent a method, whereby a functional entity, or a group of functional entities, can be considered as a processing step, or a series of processing steps, of this method. For example, the displacement detector DD can represent a displacement detection step; the object locator OL can represent an object location step.

The ultrasound imaging system UIS basically operates as follows. It is assumed that the probe PRB is in contact with the body BDY of the patient on which a suitable ointment may have been applied. The image capturing arrangement ICA produces a sequence of images IMS that are captured while the clinician inserts the needle NDL into the body BDY of the patient. To that end, the image capturing arrangement ICA applies a set of transmission signals TX to the probe PRB and processes a set of reception signals RX from the probe PRB. The set of reception signals RX comprises reflections of the transmission signals TX. These reflections occur within the body BDY of the patient. The sequence of images IMS may be so-called B- mode images, which are generated from these reception signals RX. The images may be two-dimensional or three-dimensional. The images need not necessarily comprise a visual representation of the needle NDL, or any portion thereof.

The displacement detector DD generates one or more displacement maps DM on the basis of the sequence of images IMS received from the image capturing arrangement ICA. A displacement map DM comprises respective displacement indications for respective portions of the body BDY, which are represented in the sequence of images IMS. A displacement indication may be in the form of a vector. Such a vector may have a horizontal and a vertical component corresponding with a horizontal axis and a vertical axis of an image. In case the images are three-dimensional, the vector will comprise an additional component. A displacement indication, which is associated with a particular portion of the body BDY, expresses a displacement of this portion between two images, which have been captured at different instants. This displacement will typically be a result of the needle NDL being inserted into the body BDY.

The displacement detector DD may generate respective successive displacement maps DM for respective successive images that are captured. That is, the displacement detector DD provides a displacement map DM in response to a most recent image provided by the image capturing arrangement ICA. This displacement map may express respective displacements of respective body portions with respect to an initial image. In that case, the respective displacement indications will successively increase in magnitude with each new image that is captured. This is because the needle NDL will be deeper into the body BDY with each new image that is captured. A body portion will typically undergo a displacement, which increases in magnitude as the needle NDL is inserted deeper into the body BDY. Stated otherwise, respective displacements of respective body portions become more pronounced as the needle NDL is inserted deeper into the body BDY.

The object locator OL provides an object location indication OLI on the basis of one or more displacement maps DM generated by the displacement detector DD. The object location indication OLI provides information about a current location of the needle NDL in the body BDY, or a current direction of the needle NDL in the body BDY, or both. The object locator OL effectively extracts this information from a displacement map, or a set of displacement maps DM, whichever applies. Respective displacement indications in a displacement map, which express respective displacements of respective body portions, provide information about the current location of the needle NDL, or its current direction. For example, a body portion that undergoes a relatively large displacement is typically located relatively close to the needle NDL. A line along which respective displacements have similar orientations is likely to correspond with a line along which the needle NDL has been inserted. This line typically corresponds with the direction of the needle NDL. A section along this line that exhibits a steep decrease in displacement magnitude will typically correspond with a tip portion of the needle NDL.

The object locator OL may use one or more predefined criteria for generating the object location indication OLI on the basis of one or more displacement maps DM. For example, the object locator OL may effectively search and identify an axis of symmetry in a displacement map. The axis of symmetry indicates the direction of the needle NDL. The object locator OL may further search and identify two neighboring displacement indications along the axis of symmetry one of which has a relatively large magnitude, the other having a relatively small magnitude, which is close to zero. These two neighboring displacement indications indicate the tip portion of the needle NDL. Alternatively, the object locator OL may analyze a series of successive displacement maps DM so as to search and identify a region where respective displacement indications of respective displacement maps DM remain similar in terms of orientation. This region may correspond with the direction of the needle NDL.

The object locator OL may provide respective successive object location indications OLI for respective successive displacement maps DM, which are generated for successive captured images. That is, the object locator OL provides an object location indication OLI in response to a most recent displacement map provided by the displacement detector DD. In that case, the object locator OL generates a sequence of object location indications OLI that is synchronized, as it were, with the sequence of images IMS that the image capturing arrangement ICA provides while the needle NDL is inserted into the body BDY. In a different manner of speaking, the object locator OL then provides an object location indication OLI that is continuously updated with each new image that is captured while the needle NDL is inserted into the body BDY.

The display processor DPR generates a sequence of display images DIS on the basis of the sequence of images IMS, which the image capturing arrangement ICA provides, and one or more object location indications OLI that the object locator OL provides, which may equally be in the form of a sequence as mentioned hereinbefore. The display device DPL displays the sequence of display images DIS. A display image preferably comprises a view plane from an image in the sequence of images IMS, and a visual needle indication, which is based on the object location indication OLI. The visual needle indication may comprise, for example, one or more graphic items that are overlaid on the captured image. A graphic item may convey information to the clinician by means of its position, it shape, its size, its color, or any combination of those. For example, a color-coded cursor may indicate the current location of the needle NDL in the body BDY. As another example, an arrow may indicate the direction of the needle NDL.

FIG. 2 illustrates the displacement detector DD, or rather an implementation thereof. The displacement detector DD comprises an image memory IMEM and a displacement map memory DMEM, which may physically be comprised in a single memory circuit. The displacement detector DD further comprises the following functional entities: a motion estimator ME and a displacement map accumulator DMA. As indicated hereinbefore, these functional entities may each be implemented by means of, for example, a set of instructions that have been loaded into a programmable processor. In such a software-based implementation, the motion estimator ME and the displacement map accumulator DMA may correspond with respective software modules, each of which may comprise respective sub-modules defining respective operations.

The displacement detector DD basically operates as follows. The image memory IMEM temporarily stores two or more subsequent images comprised in the sequence of images IMS that the image capturing arrangement ICA provides. At any given instant, the image memory IMEM comprises an image that the image capturing arrangement ICA has most recently provided. This image will be referred to as current image IMₖ hereinafter. The image memory IMEM further comprises an image that immediately precedes the current image IMₖ. This image will be referred to as preceding image IMₖ₋₁ hereinafter. Consequently, when the image memory IMEM receives a new image from the image capturing arrangement ICA, this new image becomes the current image IMₖ and the image that was previously the current image IMₖ becomes the preceding image IMₖ₋₁.

The motion estimator ME generates an elementary displacement map EDM for the current image IMₖ. The elementary displacement map EDM comprises respective displacement indications for respective portions of the current image IMₖ. A displacement indication indicates a displacement of the image portion concerned with respect to a corresponding image portion in the preceding image IMₖ₋₁. That is, an elementary displacement map EDM, which belongs to given image, indicates displacements that occur between that image and the immediately preceding image IMₖ₋₁. Consequently, elementary displacement maps EDM express displacements over a relatively short interval of time, namely that between two successive images. These displacements will therefore be relatively small.

The displacement map accumulator DMA generates an accumulated displacement map ADM for the current image IMₖ. The accumulated displacement map ADM comprises respective accumulated displacement indications for respective portions of the current image IMₖ. An accumulated displacement indication indicates a displacement of the image portion concerned with respect to a corresponding image portion in an initial image. That is, an accumulated displacement map ADM, which belongs to given image, indicates displacements that have occurred between that image and the initial image. The initial image may be, for example, an image that has been captured just before the needle NDL was introduced into the body BDY. Consequently, accumulated displacement maps ADM express displacements over a relatively long interval of time. These displacements will therefore be relatively large.

The displacement map accumulator DMA generates an accumulated displacement map ADM in the following fashion. The displacement map accumulator DMA stores an accumulated displacement map ADM that has most recently been generated in the displacement map memory DMEM. Let it be assumed that, at a given instant, the image memory IMEM has just received a new image from the image capturing arrangement ICA. This new image thus constitutes the current image IMₖ until a subsequent new image arrives. The motion estimator ME generates an elementary displacement map EDM for the current image IMₖ as described hereinbefore. The displacement map accumulator DMA effectively adds this elementary displacement map EDM to the accumulated displacement map ADM that is stored in the displacement map memory DMEM. This accumulated displacement map ADM belongs to the preceding image IMₖ₋₁. Accordingly, a new accumulated displacement map ADM is obtained, which belongs to the current image IMₖ. The displacement map accumulator DMA stores this accumulated displacement map ADM in the displacement map memory DMEM and may replace the generated displacement map that was previously stored therein.

A displacement map DM, which the displacement detector DD provides as mentioned hereinbefore with reference to FIG. 1, comprises an accumulated displacement map ADM. The displacement map DM may optionally further comprise a history of displacement maps HDM, which are kept in the displacement map memory DMEM. The history of displacement maps HDM may comprise respective elementary displacement maps EDM that the motion estimator ME has generated for respective images. Accordingly, when the motion estimator ME has generated the elementary displacement map EDM for the current image IMₖ, the motion estimator ME may add this elementary displacement map EDM to the history of displacement maps HDM.

The motion estimator ME may use the accumulated displacement map ADM that is stored in the displacement map memory DMEM for designating respective image portions in the preceding image IMₖ₋₁. These image portions represent corresponding respective image portions in the initial image, which have moved as a result of the needle NDL having been introduced into the body BDY. The motion estimator ME may then estimate displacements with respect to these image portions. To that end, the motion estimator ME identifies these image portions of interest in the preceding image IMₖ₋₁ on the basis of the accumulated displacement map ADM that is stored in the displacement map memory DMEM and that belongs to that image. Subsequently, the motion estimator ME searches and identifies corresponding image portions in the current image IMₖ. This results in the elementary displacement map EDM for the current image IMₖ.

In a mode of operation as described in the preceding paragraph, the displacement detector DD effectively tracks an image portion in the initial image, which portion moves throughout the sequence of images IMS that are captured while the needle NDL is introduced into the body BDY. Since an image portion in the initial image represents a particular body portion, this corresponds with tracking displacements of the body portion concerned, which are substantially caused by the needle NDL being introduced into the body BDY. The displacement detector DD tracks these displacements on an image by image basis while memorizing the location of the body portion concerned with each image. The accumulated displacement map ADM reflects this memorization.

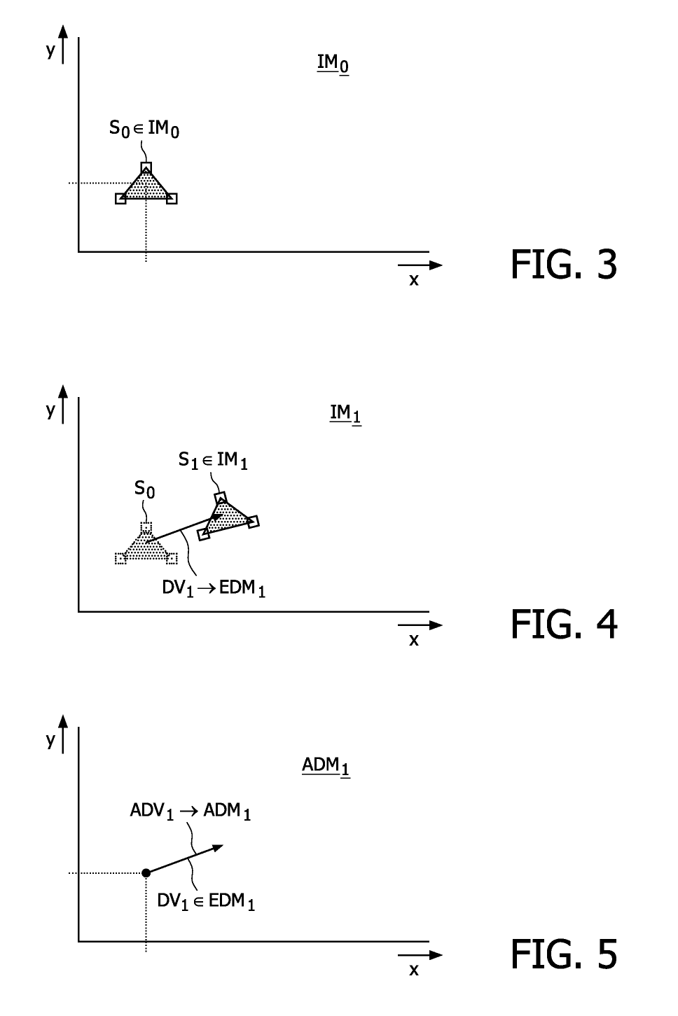

FIGS. 3-11 illustrate in more detail a manner in which the displacement detector DD may generate respective elementary displacement maps EDM and respective accumulated displacement maps ADM. This illustration involves several images that the displacement detector DD successively receives from the image capturing arrangement ICA: an initial image IM₀, a first subsequent image IM₁, a second subsequent image IM₂, and a third subsequent image IM₃.

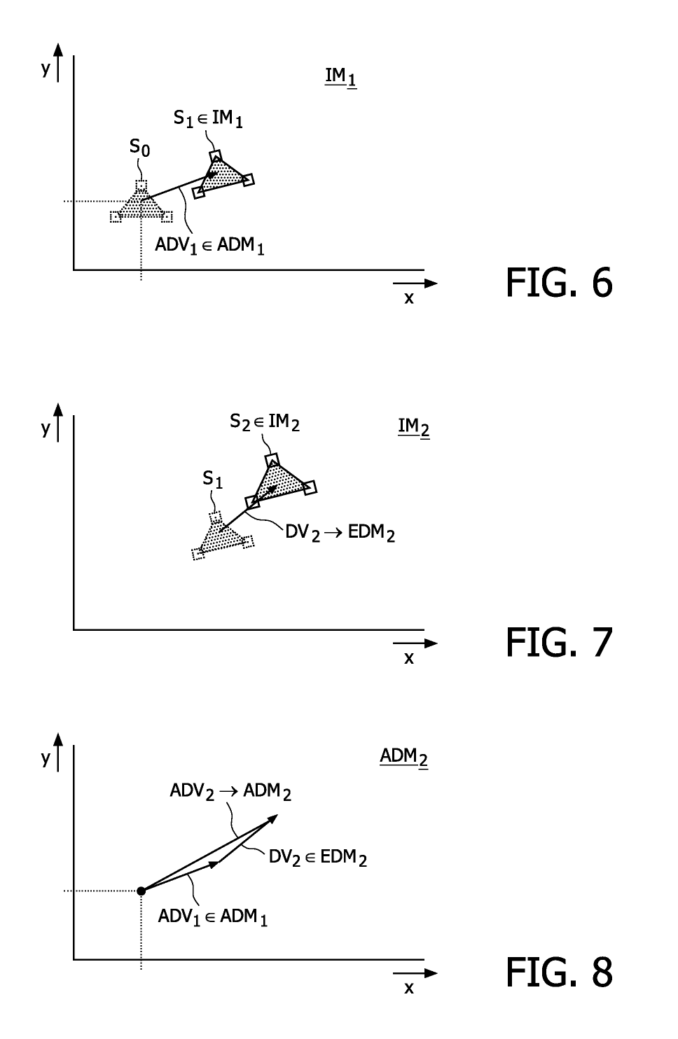

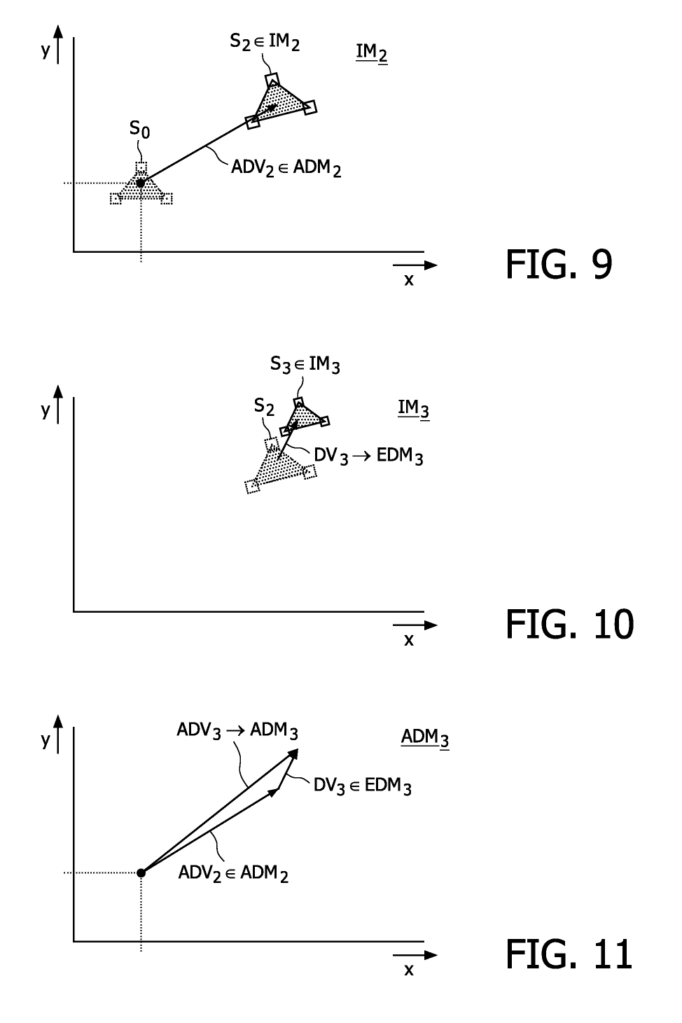

FIGS. 3-5 illustrate the manner in which the displacement detector DD may generate a first elementary displacement map EDM₁ and a first accumulated displacement map ADM₁ for the first subsequent image IM₁. FIGS. 6-8 illustrate the manner in which the displacement detector DD may generate a second elementary displacement map EDM₂ and a second accumulated displacement map ADM₂ for the second subsequent image IM₂. FIGS. 9-11 illustrate the manner in which the displacement detector DD may generate a third elementary displacement map EDM₃ and a third accumulated displacement map ADM₃ for the third subsequent image IM₃. FIGS. 3-11 each comprise a horizontal axis and a vertical axis, which represent horizontal image locations “x” and vertical image locations “y”, respectively. The images that the image capturing arrangement ICA provides are composed of graphic elements that will be referred to as texels hereinafter. In case the images are two-dimensional, a texel may correspond with a pixel. In case the images are three-dimensional, a texel may correspond with a voxel. That is, a texel represents the smallest addressable unit of the image concerned.

FIG. 3 illustrates an initial set of texels S₀ in the initial image IM₀. The motion estimator ME may designate a plurality of such texel sets, which cover, as it were, the initial image IM₀. The initial set of texels S₀ illustrated in FIG. 3 has a triangular shape and, consequently, comprises three vertices. In a motion estimation step, which serves to identify a corresponding set of texels in another image, the initial set of texels S₀ may undergo operations, such as, for example, translating, zooming, stretching, and rotating. The three vertices have respective locations with respect to each other that change as a result of the aforementioned operations. Accordingly, the three vertices, or rather a change of these, may reflect zooming, stretching, and rotating, or any combination of those. The respective locations of the three vertices of one set of texels with respect to the respective locations of the three vertices of another set of texels, may reflect a displacement between the two set of texels concerned.

FIG. 4 illustrates a first corresponding set of texels S₁ in the first subsequent image IM₁. The first corresponding set of texels S₁ corresponds with the initial set of texels S₀ in the sense that these respective sets of texels have been found to be similar. The motion estimator ME can identify the first corresponding set of texels S₁ by applying an appropriate search strategy. This search strategy may involve one or more of the aforementioned operations: zooming, stretching, and rotating. The motion estimator ME determines a first displacement vector DV₁, which represents a displacement of the first corresponding set of texels S₁ with respect to the initial set of texels S₀. The first displacement vector DV₁ constitutes an element of the first elementary displacement map EDM₁ and has a position therein, which is determined by the initial set of texels S₀ with which the first displacement vector DV₁ is associated. The motion estimator ME generates this elementary displacement map for the first subsequent image IM₁ by determining other respective first displacement vectors for other respective initial set of texels in a similar manner.

FIG. 5 illustrates a first accumulated displacement vector ADV₁, which belongs to the initial set of texels S₀ illustrated in FIG. 3. In order to illustrate this association, the first accumulated displacement vector ADV₁ has a base point in FIG. 5 that coincides with a center location of the initial set of texels S₀ in terms of horizontal image location “x” and vertical image location “y”. Since there is no accumulated displacement map that is associated with the initial image IM₀, the first accumulated displacement vector ADV₁ corresponds with the first displacement vector DV ₁. That is, the displacement map accumulator DMA makes a copy, as it were, of the first elementary displacement map EDM₁, which copy constitutes the first accumulated displacement map ADM₁.

FIG. 6-8 illustrate operations that the displacement detector DD carries out for the purpose of generating the second elementary displacement map EDM₂ and the second accumulated displacement map ADM₂. The displacement detector DD carries out these operations when the second subsequent image IM₂ has arrived and is present in the image memory IMEM. The second subsequent image IM₂ then constitutes the current image as defined hereinbefore, and the first subsequent image IM₁ then constitutes the preceding image.

FIG. 6 illustrates that the motion estimator ME designates a set of texels in the first subsequent image IM₁ that corresponds with initial set of texels S₀ in the initial image IM₀ illustrated in FIG. 3. The motion estimator ME may designate this set of texels, which is the first corresponding set of texels S₁, on the basis of the first accumulated displacement map ADM₁, which belongs to the first subsequent image IM₁. The first accumulated displacement comprises the first accumulated displacement vector ADV₁ illustrated in FIG. 5, which vector belongs to the initial set of texels S₀ illustrated in FIG. 3.

FIG. 7 illustrates that the motion estimator ME identifies a second corresponding set of texels S₂ in the second subsequent image IM₂. The second corresponding set of texels S₂ is a set of texels in the second subsequent image IM₂ that best matches, as it were, the first corresponding set of texels S₁. Since the first corresponding set of texels S₁ best matches the initial set of texels S₀, the second corresponding set of texels S₂ will also match with the initial set of texels S₀. The motion estimator ME determines a second displacement vector DV₂, which represents a displacement of the second corresponding set of texels S₂ with respect to the first corresponding set of texels S₁. The second displacement vector DV₂ constitutes an element of the second elementary displacement map EDM₂ and has a position therein, which is determined by the initial set of texels S₀ with which the second displacement vector DV₂ is associated.

FIG. 8 illustrates a second accumulated displacement vector ADV₂, which belongs to the initial set of texels S₀ illustrated in FIG. 3. The displacement map accumulator DMA generates the second accumulated displacement vector ADV₂ by adding the second displacement vector DV₂, which is obtained as illustrated in FIG. 7, to the first accumulated displacement vector ADV₁, which was previously established for the initial set of texels S₀ as illustrated in FIGS. 3-5. That is, the second accumulated displacement vector ADV₂ is a vectorial sum of the first accumulated displacement vector ADV₁, which is present in the first cumulative displacement map, and the second displacement vector DV₂. The displacement map accumulator DMA may thus generate the second accumulated displacement map ADM₂ by determining other respective second accumulated displacement vectors for other respective initial set of texels in a similar manner.

FIG. 9-11 illustrate operations that the displacement detector DD carries out for the purpose of generating the third elementary displacement map EDM₃ and the third accumulated displacement map ADM₃. The displacement detector DD carries out these operations when the third subsequent image IM₃ has arrived and is present in the image memory IMEM. The third subsequent image IM₃ then constitutes the current image as defined hereinbefore, and the second subsequent image IM₂ then constitutes the preceding image.

FIG. 9 illustrates that the motion estimator ME designates a set of texels in the second subsequent image IM₂ that corresponds with initial set of texels S₀ in the initial image IM₀ illustrated in FIG. 3. The motion estimator ME may designate this set of texels, which is the second corresponding set of texels S₂, on the basis of the second accumulated displacement map ADM₂, which belongs to the second subsequent image IM₂. The second accumulated displacement comprises the second accumulated displacement vector ADV₂ illustrated in FIG. 8, which vector belongs to the initial set of texels S₀ illustrated in FIG. 3.

FIG. 10 illustrates that the motion estimator ME identifies a third corresponding set of texels S₃ in the third subsequent image IM₃. The third corresponding set of texels S₃ is a set of texels in the third subsequent image IM₃ that best matches, as it were, the second corresponding set of texels S₂. Since the second corresponding set of texels S₂ matches with the initial set of texels S₀, the third corresponding set of texels S₃ will also match with the initial set of texels S₀. The motion estimator ME determines a third displacement vector DV₃, which represents a displacement of the third corresponding set of texels S₃ with respect to the second corresponding set of texels S₂. The third displacement vector DV₃ constitutes an element of the third elementary displacement map EDM₃ and has a position therein, which is determined by the initial set of texels S₀ with which the third displacement vector DV₃ is associated.

FIG. 11 illustrates a third accumulated displacement vector ADV₃, which belongs to the initial set of texels S₀ illustrated in FIG. 3. The displacement map accumulator DMA generates the third accumulated displacement vector ADV₃ by adding the third displacement vector DV₃, which is obtained as illustrated in FIG. 10, to the second accumulated displacement vector ADV₂, which was previously established for the initial set of texels S₀ as illustrated in FIGS. 6-8. That is, the third accumulated displacement vector is a vectorial sum of the second accumulated displacement vector ADV₂, which is present in the second accumulated displacement map ADM₂, and the third displacement vector DV₃. The displacement map accumulator DMA may thus generate the third accumulated displacement map ADM₃ by determining other respective third accumulated displacement vectors for other respective initial set of texels in a similar manner.

The displacement detector DD may continue carrying out operations as illustrated in FIGS. 3-11 so as to generate respective further elementary displacement maps EDM and respective further accumulated displacement maps ADM for respective further images that the image capturing arrangement ICA provides. That is, the displacement detector DD may provide an elementary displacement map EDM and an accumulated displacement map ADM for each further image that is captured while the needle is inserted into the body as illustrated in FIG. 1.

With each further accumulated displacement map ADM that the displacement detector DD generates, the respective accumulated displacement vectors will grow in magnitude, as it were. Consequently, differences between respective accumulated displacement vectors typically become more pronounced with each image that the displacement detector DD processes. In a manner of speaking, displacement contrast will successively increase.

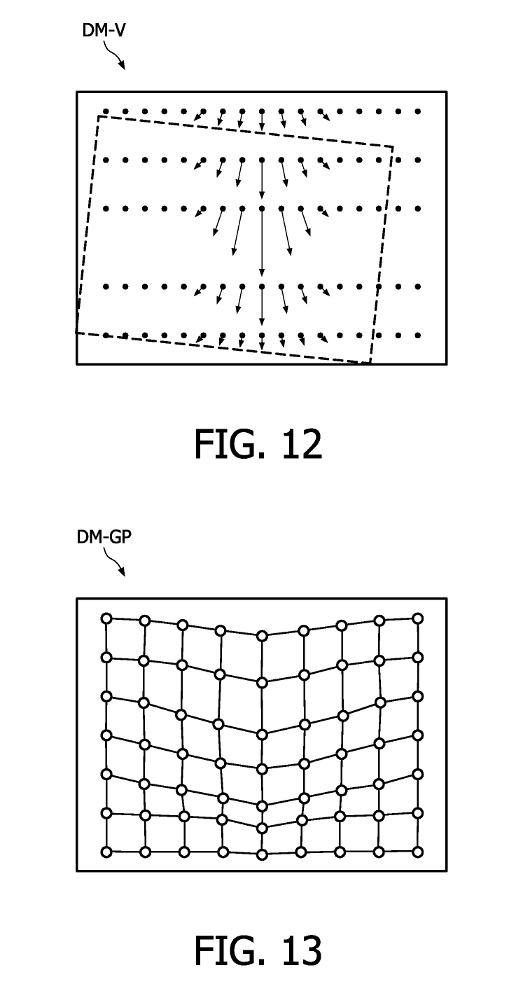

FIG. 12 illustrates a vector-based displacement map DM-V that the displacement detector DD may provide. The vector-based displacement map DM-V corresponds with an accumulated displacement map ADM that has been obtained as described hereinbefore with reference to FIGS. 3-11. The vector-based displacement map DM-V comprises respective accumulated displacement vectors for respective initial sets of texels. An accumulated displacement vector reflects a displacement that a body portion represented by the initial set of texels concerned has undergone as a result of the needle having been introduced into the body.

The object locator OL illustrated in FIG. 1 can provide an object location indication OLI on the basis of the vector-based displacement map DM-V illustrated in FIG. 12. The object locator OL may do so in various different manners as discussed hereinbefore with reference to FIG. 1. For example, the object locator OL may search and identify an axis of symmetry in the vector-based displacement map DM-V, which indicates the direction of the needle NDL. For reasons of convenience, the axis of symmetry is horizontally centered in FIG. 12. The axis of symmetry indicates the direction of the needle NDL. In practice, the axis of symmetry may not be centered due to, for example, a misalignment of the probe illustrated in FIG. 1 with respect to the needle. FIG. 12 illustrates such a misalignment by means of a rectangle with broken border lines. This rectangle can be regarded as representing a displacement map that is obtained in practice, in which the axis of symmetry need not necessarily be horizontally centered or aligned with a border of the displacement map.

The object locator OL may further search and identify a steep decrease in magnitude of accumulated displacement vectors along the axis of symmetry. The steep decrease of interest occurs where an accumulated displacement vector has an almost zero magnitude, whereas this vector is preceded by an accumulated displacement vector that has a significant magnitude. Such a steep decrease indicates the tip portion of the needle NDL, which is at the center bottom in FIG. 12.

It should be noted that there are numerous techniques for identifying an axis of symmetry in a displacement map, such as the vector-based displacement map DM-V illustrated in FIG. 12. An example of such a technique is briefly indicated in what follows. A grid of texel locations, which can be found in image, may be defined. Respective grid points may correspond with respective initial sets of texels for which respective accumulated displacement vectors have been generated as described hereinbefore. Respective counters are assigned to respective grid points. Initially, the respective counters are each set to zero. For each grid point, a line is drawn following the direction of the accumulated displacement vector that belongs to the grid point concerned. The counter of a grid point is incremented by one unit for each line that traverses a predefined zone around the grid point. Counters that are on the axis of symmetry will produce relatively high count values. The axis of symmetry may be visualized, for example, by associating gray values with count values. White may represent a zero count value; black may represent a maximum count value. Such a grayscale map has a contrast that may be increased by means of post-processing, which may comprise operations such as, for example, noise reduction, line regression, or thresholding, or any combination of those. The finer the aforementioned grid of texel locations is, the more precise the needle direction can be indicated.

The description hereinbefore with reference to FIGS. 3-12 concerns an example in which the accumulated displacement map ADM comprises vectors, which are updated with each motion estimation that is carried out for each new image. That is, long-term displacements are expressed by means of vectors, by successively summing short-term vectors that express displacements between two successive images. The term “long-term” relates to a time interval that covers multiple successive images provided by the image capturing arrangement ICA. However, long-term displacements may be expressed differently.

For example, long-term displacements may be expressed by means of grid points. A grid of equidistantly spaced points may be defined for an initial image. A grid point corresponds with a particular location in the initial image, which may be expressed by means of a set of coordinates such as, for example, (x,y) in case of a two-dimensional image or (x,y,z) in case of a three-dimensional image. The grid point moves with each motion estimation that the motion estimator ME illustrated in FIG. 2 carries out. Accordingly, the displacement detector DD generates a map of grid points for each new image in the sequence of images IMS. The map of grid points that is generated for a particular image is an updated version of the map of grid points that was generated for a preceding image. To that end, the displacement detector DD may apply the elementary displacement map EDM that is generated for the image concerned, to the map of grid points that was generated for the preceding image. The accumulated displacement indication map ADM illustrated in FIG. 2 may thus be in the form of a map of grid points, which is updated on an image by image basis.

FIG. 13 illustrates a grid point-based displacement map DM-GP that the displacement detector DD may provide. The grid point-based displacement map DM-GP comprises respective grid points, which have moved with respect to those defined for the initial image. The grid of equidistantly spaced points for the initial image has become deformed, as it were, because respective grid points have experienced respective displacements. The grid point-based displacement map DM-GP thus reflects respective displacements that respective body portions have undergone as a result of the needle having been introduced into the body.

In order to obtain the grid point-based displacement map DM-GP illustrated in FIG. 13, the displacement detector DD illustrated in FIGS. 1 and 2 may operate in a fashion that differs from that described hereinbefore with reference to FIGS. 3-12. For example, the displacement detector DD may carry out a motion estimation for a pair of temporally neighboring images in the following fashion. The displacement detector DD may designate respective sets of texels in one image of the pair in accordance with a standard pattern, which need not depend on any displacement history. The displacement detector DD determines a motion vector for each set of texels, by identifying a similar set of texels in the other image. Accordingly, a map of motion vectors is obtained, which is functionally equivalent to the elementary displacement map EDM in the description with reference to FIGS. 3-12. The motion vectors may be equidistantly spaced, in accordance with the standard pattern that was used to designate respective sets of texels.

The displacement detector DD may apply a map of motion vectors to a map of grid points, which is functionally equivalent to the accumulated displacement map ADM in the description with reference to FIGS. 3-12. The grid points will typically not be equidistantly spaced, as illustrated in, for example, FIG. 13. That is, a grid point need not necessarily coincide with a motion vector. However, the grid point will be surrounded by motion vectors. The grid point may then undergo a displacement that is defined by weighted combination of surrounding motion vectors. The closer a motion vector is to the grid point, the higher the weight that is given to that motion vector. Accordingly, the map of motion vectors causes respective grid points to undergo respective displacements so as to obtain an updated version of the map of grid points. Long-term displacement tracking is achieved by updating the map of grid points on the basis of respective maps of motion vectors, which are determined for successive images.

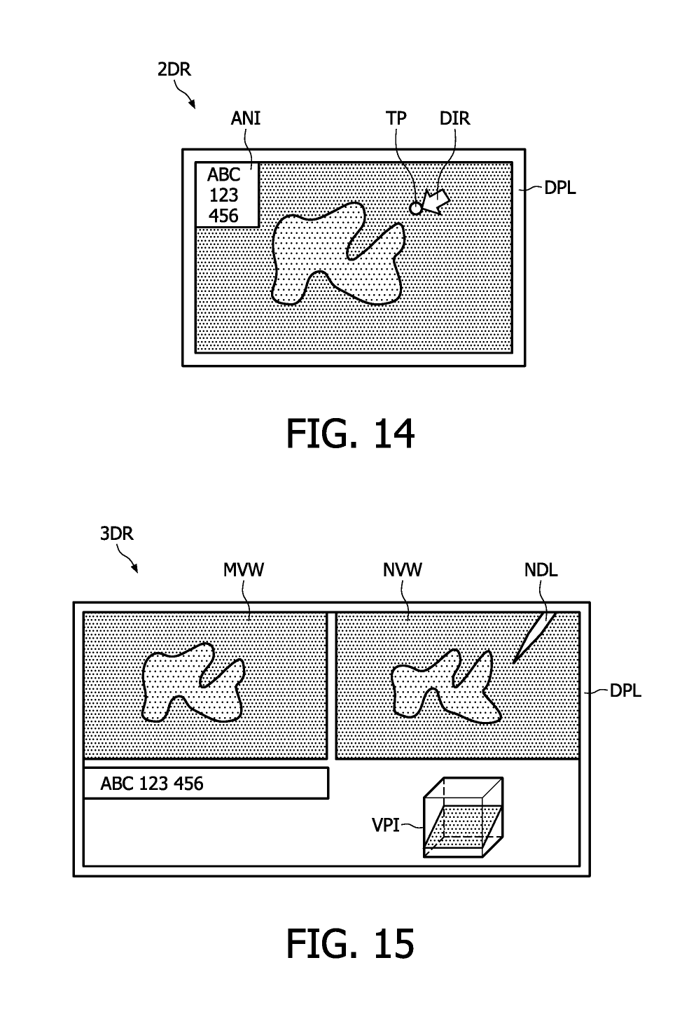

FIG. 14 illustrates a 2-D mode display image 2DR, which the display processor DPR illustrated in FIG. 1 may provide based on a two-dimensional ultrasound scan. The display image comprises a captured image, which represents a region of interest within the body BDY. The display image further comprises visual indications pertaining to the current location of the needle, or its current direction, or both. These visual indications are based on the object location indication OLI provided by the object locator OL illustrated in FIG. 1. For example, the display image may comprise a direction indication DIR and a tip location indication TP as illustrated in FIG. 14. This is merely an illustration of one among numerous possible variants. The direction indication may be in the form of, for example, a straight line that extends from a graphic item that represents a tip location. The display image may further comprise a section ANI with alphanumerical information, which may include information pertaining to the location and direction of the needle NDL.

FIG. 15 illustrates a 3-D mode display image 3DR, which the display processor DPR illustrated in FIG. 1 may provide based on a three-dimensional ultrasound scan. The display image comprises a main view MVW and a needle view NVW. The main view MVW may be a three-dimensional representation of the region of interest, or an arbitrary view plane in a volume of data that has been acquired. The needle view NVW corresponds with a view plane in which the needle NDL lies. The display processor DPR may automatically identify this view plane on the basis of the object location indication OLI that the object locator OL provides. The display image may further comprise a view plane indication that indicates the location of the view plane in which the needle NDL lies.

### Concluding Remarks:

The detailed description hereinbefore with reference to the drawings is merely an illustration of the invention and the additional features, which are defined in the claims. The invention can be implemented in numerous different ways. In order to illustrate this, some alternatives are briefly indicated.

The invention may be applied to advantage in numerous types of products or methods related to ultrasound imaging. The body, into which the object is introduced while ultrasonic imaging in accordance with the invention is carried out, need not necessarily be of a biological nature. For example, the invention may be applied to operate on composite materials. The object that is introduced need not necessarily be a needle. For example, the invention may be applied to advantage for inserting a sensor or an antenna into a body. The antenna may be used, for example, for clinical purposes.

There are numerous ways of generating a map of displacement indications from a sequence of ultrasound images. In this respect it should be noted that there a is vast literature on motion estimation, describing numerous different techniques, which may be applied to implement the invention. For example, a block matching algorithm intended for MPEG encoding may be used. Feature-based algorithms, as well as optical flow algorithms, phase correlation algorithms, to name a few others, may equally be used.

There are numerous ways of providing an indication relating to the location of the object in the body on the basis of displacement indications. For example, an indication may be derived from a recorded history of displacement indications, which may be reflected in a map. A line along which there is a coherent evolution in displacement indications, may indicate a direction in which the object moves.

The term “image” should be understood in a broad sense. The term includes any collection of data or signals that may be visually represented either directly or through appropriate processing of the collection of data or signals concerned. The term image includes entities, such as, for example, picture, frame, or field. The term image comprises two-dimensional as well as three-dimensional representations.

In broad terms, there are numerous ways of implementing functional entities by means of hardware or software, or a combination of both. Although software-based implementations as indicated in the detailed description are generally preferred, hardware-based implementations are by no means excluded. For example, any functional entity described hereinbefore may equally be implemented by means of a dedicated circuit, which has a particular topology defining one or more operations that the functional entity concerned carries out. Hybrid implementations are also possible in the sense that a system, or a functional entity comprises therein, comprises one or more dedicated circuits as well as one or more suitably programmed processors.

Although a drawing shows different functional entities as different blocks, this by no means excludes implementations in which a single entity carries out several functions, or in which several entities carry out a single function. In this respect, the drawings are very diagrammatic. For example, referring to FIG. 1, a single programmable circuit may be programmed to carry out operations belonging to the controller CTRL, the displacement detector DD, and the object locator OL. As another example, referring to FIG. 2, the motion estimator ME and the displacement map accumulator DMA may be comprised in a single integrated circuit, which may further comprise the image memory IMEM or the displacement map memory DMEM, or both memories.

There are numerous ways of storing and distributing a set of instructions, that is, software, which allows a programmable circuit to operate in accordance with the invention. For example, software may be stored in a suitable medium, such as an optical disk or a memory circuit. A medium in which software stored may be supplied as an individual product or together with another product, which may execute software. Such a medium may also be part of a product that enables software to be executed. Software may also be distributed via communication networks, which may be wired, wireless, or hybrid. For example, software may be distributed via the Internet. Software may be made available for download by means of a server. Downloading may be subject to a payment.

The remarks made herein before demonstrate that the detailed description with reference to the drawings, illustrate rather than limit the invention. There are numerous alternatives, which fall within the scope of the appended claims. Any reference sign in a claim should not be construed as limiting the claim. The word “comprising” does not exclude the presence of other elements or steps than those listed in a claim. The word “a” or “an” preceding an element or step does not exclude the presence of a plurality of such elements or steps. The mere fact that respective dependent claims define respective additional features, does not exclude a combination of additional features, which corresponds to a combination of dependent claims.

1. A method of ultrasound imaging comprising:
an image capturing step in which a sequence of ultrasound images (IMS) of a body (BDY) is captured while an object (NDL) is introduced into the body;
a displacement detection step in which a map of displacement indications (DM) are generated from the sequence of ultrasound images, a displacement indication relating to a particular portion of the body and indicating a displacement that the portion has undergone; and
an object location step in which an indication (OLI) relating to the location of the object in the body is provided on the basis of the map of displacement indications.

2. A method of ultrasound imaging according to claim 1, comprising:
a display processing step in which a display image (DIS) is formed that comprises an ultrasound image and a visual indication (TP, DIR) that is based on the indication (OLI) relating to the location of the object in the body, which is provided in the object location step.

3. A method of ultrasound imaging according to claim 1, the object location step comprising a direction identification sub-step in which an axis of symmetry is identified in the map of displacement indications (DM).

4. A method of ultrasound imaging according to claim 3, comprising:
a display processing step in which a display image (2DR) is formed that comprises an ultrasound image and a visual indication of a direction (DIR) in which the object (NDL) moves within the body (BDY), the visual indication being based on the axis of symmetry, which has been identified in the direction identification sub-step.

5. A method of ultrasound imaging according to claim 3, the object location step comprising a tip portion identification sub-step in which a steep decrease in magnitude of displacement indications along the axis of symmetry is identified.

6. A method of ultrasound imaging according to claim 5, comprising:
a display processing step in which a display image (2DR) is formed that comprises an ultrasound image and a visual indication of a tip portion (TP) of the object (NDL), the visual indication being based on the steep decrease in magnitude of displacement indications along the axis of symmetry, which has been identified in the tip portion identification sub-step.

7. A method of ultrasound imaging according to claim 1, wherein the image capture in step involves a three-dimensional scan of the body that produces volume data, the method comprising:
a view plane generation step in which a view plane (NVW) that coincides with the object (NDL) introduced into the body (BDY) is generated from the volume data on the basis of the indication (OLI) relating to the location of the object in the body, which is provided in the object location step; and
a display processing step in which a display image (3DR) is formed that comprises the view plane.

8. A method of ultrasound imaging according to claim 1, the displacement detection step comprising:
a motion estimation step in which a map of elementary displacement indications (EDM) is generated from a pair of ultrasound images (IMₖ, IMₖ₋₁), which are temporarily neighboring, an elementary displacement indication linking a particular location in one image of the pair to a particular location in the other image; and
a displacement map accumulation step in which a map of accumulated displacement indications (ADM) is generated on the basis of respective maps of elementary displacement indications generated from respective pairs of ultrasound images, an accumulated displacement indication corresponding to a sum of respective elementary displacement indications that link respective locations in respective images.

9. A method of ultrasound imaging according to claim 8, the motion estimation step and the displacement map accumulation step being carried out on an image by image basis, whereby the displacement map accumulation step comprises:
a memory read sub-step in which a recent version of the map of accumulated displacement indications (ADM), which was previously generated, is read from a memory (DMEM);
an accumulation step in which respective elementary displacement indications (EDM) that are generated from a pair of ultrasound images (IMₖ, IMₖ₋₁) are applied to corresponding respective accumulated displacement indications comprised in the map of accumulated displacement indications, which has been read from the memory, so as to obtain an updated version of the map of accumulated displacement indications; and
a memory write step in which the updated version of the map of accumulated displacement indications is written into a memory.

10. A method of ultrasound imaging according to claim 8, whereby, in the displacement map accumulation step, the accumulated displacement indications are expressed as respective points associated with respective locations in an initial image, the respective points being shifted in terms of image location as a result of respective elementary displacement indications that have been established in the motion estimation step.

11. An ultrasound imaging system (UIS), comprising:
an image capturing arrangement (ICA) adapted to capture a sequence of ultrasound images (IMS) of a body (BDY) while an object (NDL) is introduced into the body;
a displacement detector (DD) adapted to generate a map of displacement indications (DM) from the sequence of ultrasound images, a displacement indication relating to a particular portion of the body and indicating a displacement that the portion has undergone; and
an object locator (OL) adapted to provide an indication (OLI) relating to the location of the object in the body on the basis of the map of displacement indications.

12. A computer program product that comprises a set of instructions, which when loaded into a programmable processor, causes the programmable processor to carry out the method as claimed in claim 1.
